(19) United States
(12) Patent Application Publication
(10) Pub. No.: US 2026/0248493 A1
(43) Pub. Date: Aug. 27, 2026

(54) CLOSURE DEVICES

(71) Applicant: Boston Scientific Scimed, Inc., Maple Grove, MN (US)

(72) Inventors: Blaine Andrew Schneider, Corcoran, MN (US); Kristen Lipschultz, Minneapolis, MN (US); Bradley Erik Steele, Plymouth, MN (US); Shanna Klein, Plymouth, MN (US); Sunil Shah, Fremont, CA (US)

(73) Assignee: Boston Scientific Scimed, Inc., Maple Grove, MN (US)

(21) Appl. No.: 19/548,064

(22) Filed: Feb. 24, 2026

Related U.S. Application Data
(60) Provisional application No. 63/762,727, filed on Feb. 25, 2025.

(51) Int. Cl. A61B 17/00; A61B 90/00

(52) U.S. Cl. CPC A61B 17/0057; A61B 2017/00557; A61B 2017/00592; A61B 2017/00601; A61B 2017/00615; A61B 2017/00619; A61B 2017/00623; A61B 2017/00884; A61B 2090/3925; A61B 2090/3966

(57) ABSTRACT

Vascular closure devices as well as methods for making and using vascular closure devices are disclosed. An example vascular closure device may include an intravascular sealing member configured to be disposed within a blood vessel. The vascular closure device may also include an extravascular hemostatic member configured to be disposed adjacent to an exterior of the blood vessel. The vascular closure device may also include an extravascular sealing member configured to be disposed along an outer surface of the extravascular hemostatic member. A suture may be coupled to the intravascular sealing member and the extravascular sealing member. At least one of the intravascular sealing member, the extravascular hemostatic member, the extravascular sealing member, and the suture may include a radiopaque material.

### CROSS-REFERENCE TO RELATED APPLICATIONS

This application claims the benefit of priority under 35 U.S.C. §119 of U.S. Provisional Application No. 63/762,727, filed Feb. 25, 2025, the entire disclosure of which is hereby incorporated by reference.

### TECHNICAL FIELD

The present disclosure pertains to medical devices, and methods for manufacturing medical devices. More particularly, the present disclosure pertains to vascular closure devices.

### BACKGROUND

A wide variety of medical devices have been developed for medical use, and more specifically for intravascular use. Some of these devices include guidewires, catheters, vascular closure devices, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A vascular closure device is disclosed. The vascular closure device comprises: an intravascular sealing member configured to be disposed within a blood vessel; an extravascular hemostatic member configured to be disposed adjacent to an exterior of the blood vessel; an extravascular sealing member configured to be disposed along an outer surface of the extravascular hemostatic member; a suture coupled to the intravascular sealing member and the extravascular sealing member; and wherein at least one of the intravascular sealing member, the extravascular hemostatic member, the extravascular sealing member, and the suture includes a radiopaque material.

Alternatively or additionally to any of the embodiments above, the intravascular sealing member includes the radiopaque material.

Alternatively or additionally to any of the embodiments above, the radiopaque material is disposed along an outer surface of the intravascular sealing member.

Alternatively or additionally to any of the embodiments above, the radiopaque material is embedded within the intravascular sealing member.

Alternatively or additionally to any of the embodiments above, the suture includes the radiopaque material.

Alternatively or additionally to any of the embodiments above, further comprising a delivery sheath and a delivery arm disposed within the delivery sheath for delivering the vascular closure device to a vessel opening.

Alternatively or additionally to any of the embodiments above, the delivery arm includes a radiopaque material.

Alternatively or additionally to any of the embodiments above, further comprising a balloon coupled to the delivery arm.

Alternatively or additionally to any of the embodiments above, the balloon includes a radiopaque material.

Alternatively or additionally to any of the embodiments above, further comprising a locking member coupled to the suture.

Alternatively or additionally to any of the embodiments above, the locking member includes a radiopaque material.

A vascular closure device is disclosed. The vascular closure device comprises: a suture; an intravascular sealing member coupled to the suture, the intravascular sealing member configured to be disposed within a blood vessel; an extravascular sealing member coupled to the suture, the extravascular sealing member configured to be disposed adjacent to an outer surface of the blood vessel; an extravascular hemostatic member coupled to the suture and disposed between the intravascular sealing member and the extravascular sealing member; and wherein the suture, the intravascular sealing member, or both include an imaging member configured to enhance visualization thereof.

Alternatively or additionally to any of the embodiments above, the imaging member includes a radiopaque material disposed along the intravascular sealing member.

Alternatively or additionally to any of the embodiments above, the imaging member includes a radiopaque material embedded within the intravascular sealing member.

Alternatively or additionally to any of the embodiments above, the imaging member includes a radiopaque material incorporated into the suture.

Alternatively or additionally to any of the embodiments above, the imaging member includes an echogenic material disposed along or embedded within the intravascular sealing member.

Alternatively or additionally to any of the embodiments above, the imaging member includes an echogenic surface formed along the intravascular sealing member.

Alternatively or additionally to any of the embodiments above, the imaging member includes a radiopaque balloon disposed adjacent to the intravascular sealing member.

Alternatively or additionally to any of the embodiments above, further comprising a radiopaque locking member coupled to the suture.

A system for closing a vascular opening is disclosed. The system comprises: a delivery sheath; a delivery arm slideably disposed within the delivery sheath; and a closure device coupled to the delivery arm, the closure device comprising: a suture, an intravascular sealing member coupled to the suture, the intravascular sealing member configured to be disposed within a blood vessel, an extravascular sealing member coupled to the suture, the extravascular sealing member configured to be disposed adjacent to an outer surface of the blood vessel, an extravascular hemostatic member coupled to the suture and disposed between the intravascular sealing member and the extravascular sealing member, and wherein the suture, the intravascular sealing member, or both include an imaging member configured to enhance visualization thereof.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a perspective view of an example vascular closure device.

FIG. 2 is a side view of an example vascular closure device disposed adjacent to a blood vessel.

FIG. 3 is a top view of a portion of an example vascular closure device.

FIG. 4 is a top view of a portion of an example vascular closure device.

FIG. 5 is a top view of a portion of an example vascular closure device.

FIG. 6 is a top view of a portion of an example vascular closure device.

FIG. 7 is a cross-sectional view of a portion of an example vascular closure device.

FIG. 8 is a side view of an example vascular closure device disposed adjacent to a blood vessel.

FIG. 9 is a side view of a portion of an example vascular closure device.

FIG. 10 is a top view of a portion of an example vascular closure device.

FIG. 11 is a side view of an example vascular closure device disposed adjacent to a blood vessel.

FIG. 12 is a side view of an example vascular closure device disposed adjacent to a blood vessel.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Percutaneous vascular procedures are used in conjunction with a number of different medical interventions. Such procedures include forming an opening through the skin and into a blood vessel. During the medical intervention procedure and specifically upon its conclusion, the opening needs to be managed and ultimately closed. A number of different vascular closure devices have been developed for helping to manage and close the opening. In some instances, it may be challenging to perform a vascular closure intervention at least because a user may not be able to visualize the vascular closure device at the access site. Such challenges may be exacerbated when the access site is at the carotid artery. Disclosed herein are vascular closure devices with improved visualization. Such devices may be useful at a number of different access sites including the carotid artery.

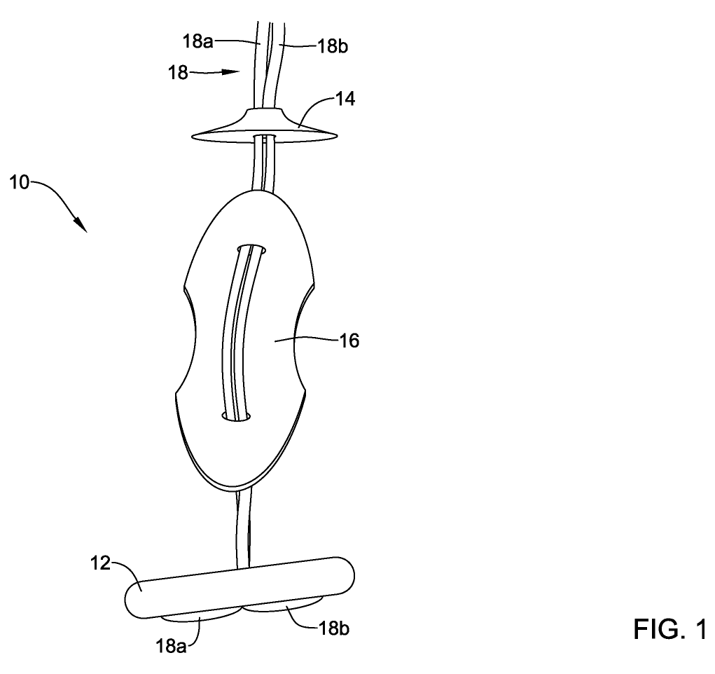

FIG. 1 illustrates an example vascular closure device 10. The vascular closure device 10 may include an intravascular sealing member 12, an extravascular sealing member 14, and an extravascular hemostatic member 16. A suture 18 may be coupled to one or more of the components of the vascular closure device 10 (e.g., the intravascular sealing member 12, the extravascular sealing member 14, and the extravascular hemostatic member 16). The extravascular hemostatic member 16 may comprise a hemostatic sponge, foam, sheet, and/or the like. Some example materials for the extravascular hemostatic member 16 may include gelatin, collagen, polyvinyl acetate (PVA), oxidized regenerated cellulose, polyethylene glycol (PEG), carboxymethyl cellulose, hydroxyethyl cellulose and amylopectin, fibrinogen/thrombin (additives to help with clot formation), other bioresorbable hemostatic materials or clotting agents, and/or the like. The suture 18 may include loops or strands 18a, 18b that engage, for example, the intravascular sealing member 12.

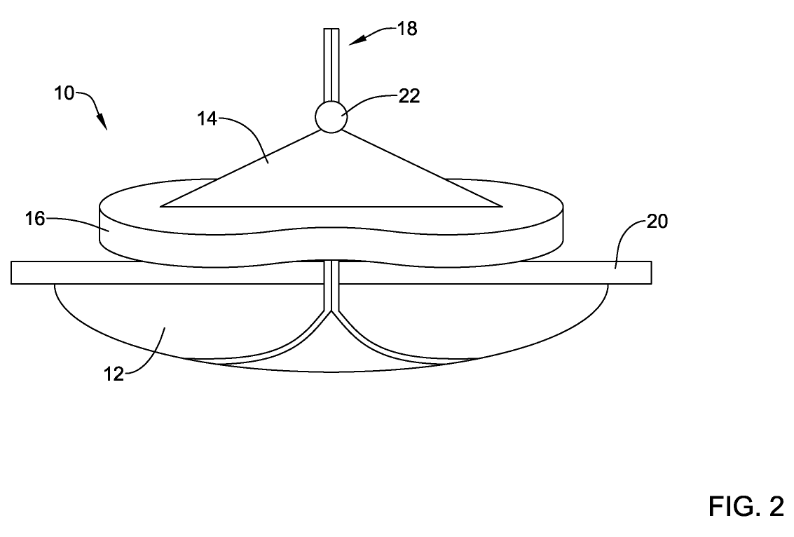

In general, the intravascular sealing member 12 may be configured to be disposed within a blood vessel 20 as shown in FIG. 2. In at least some instances, the blood vessel 20 may be a carotid artery or adjacent vessel. The extravascular sealing member 14 may be configured to be disposed along an outer surface of the blood vessel 20. The extravascular hemostatic member 16 may be configured to be disposed adjacent to an exterior of the blood vessel 20 and, in general, between the intravascular sealing member 12 and the extravascular sealing member 14. The intravascular sealing member 12 and the extravascular sealing member 14 (e.g., along with the extravascular hemostatic member 16) may be brought together to close the opening in the blood vessel 20. In some instances, a locking member 22 may be disposed along the suture 18, for example for securing the position of the intravascular sealing member 12 relative to the extravascular sealing member 14.

At least one of the intravascular sealing member 12, the extravascular sealing member 14, the extravascular hemostatic member 16, and the suture 18 includes an imaging member configured to enhance visualization thereof. For example, at least one of the intravascular sealing member 12, the extravascular sealing member 14, the extravascular hemostatic member 16, and the suture 18 may include a radiopaque material, an echogenic material, combinations thereof, and/or the like. This may useful when managing/sealing/closing a number of different vascular openings. For example, managing/sealing/closing openings into the carotid artery or adjacent vessels may be challenging and could result in significant blood loss if the vascular closure procedure is not performed with suitable precision. Thus, by enhancing the visualization of the vascular closure device 10, it may be possible to improve the efficiency and effectiveness of vascular closure processes, particularly when used to seal/close openings into the carotid artery or adjacent vessels. It can be appreciated that visually identifying the extravascular sealing member 14 and/or the extravascular hemostatic member 16 (e.g., external components) in addition to the intravascular sealing member 12 and/or the suture 18 (e.g., internal components) may be desirable, for example, by providing the ability to confirm that these components are properly placed (e.g., the external components are not in the lumen of the vessel).

When the vascular closure device 10 includes a radiopaque material, the radiopaque material may be incorporated into one or more components of the vascular closure device 10 so that the component can be visualized using conventional fluoroscopy. This may include disposing a radiopaque material or member along one or more of the components of the vascular closure device 10, embedding a radiopaque material or member into one or more of the components of the vascular closure device 10, incorporating a radiopaque material or member into one or more of the components of the vascular closure device 10, using additional radiopaque components, combinations thereof, and/or the like. Some example radiopaque materials may include barium chloride, barium sulfate, bismuth, bismuth subcarbonate, bismuth oxychloride, bismuth trioxide, calcium tungstate, gold, gadolinium, iridium, palladium, platinum, rhenium, tantalum, tungsten, ytterbium trifluoride, zirconium dioxide, radiopaque nanoparticles (e.g., barium sulfate, gold, iodixanol, iron (II/III) oxide, other formulations such as LoPro Clear™ available from Foster’s corporation, etc.), bioresorbable radiopaque materials (e.g., bromine, calcium, calcium carbonate, iodine, iodixanol, iron, magnesium, magnesium alloys, Mg₃Zn₆Y, Magnesium alloy WE-43 available from Smiths Metal, combinations thereof, and/or the like), polydiolcitrate-MoS₂ composite materials, poly-L-lactic acid, loaded inks (e.g., tungsten loaded inks), radiopaque material flakes, radiopaque marker bands, other materials disclosed herein, and/or the like. These are just examples. Some example echogenic material may include bioabsorbable materials (e.g., including those disclosed herein), materials with surface roughness, materials with surface patterns (e.g., dimples, striations, etc.), materials with small/micro air bubbles, and/or the like.

The adding/incorporating a radiopaque material into one or more components of the vascular closure device 10 may be accomplished in a number of different ways. For example, components such as the intravascular sealing member 12 and/or the extravascular sealing member 14 may be formed from a polymeric material. A radiopaque material may be loaded/doped/blended/impregnated into the polymer material (e.g., including different levels of loading to customize the desired level of visibility/imageability). When it is desired to have one or more component be bioresorbable, bioresorbable radiopaque materials may be incorporated into one or more components of the vascular closure device 10, which may make that component and/or the complete vascular closure device fully resorbable. In the case where the suture 18 includes a radiopaque material, the radiopaque material may comprise a radiopaque strand or filament that is woven, threaded, braided, or otherwise incorporated into the suture 18.

For the purposes of this disclosure, it can be understood that vascular closure devices 10 are contemplated where (a) only the intravascular sealing member 12 may include a radiopaque material, (b) only the extravascular sealing member 14 may include a radiopaque material , (c) only the extravascular hemostatic member 16 includes a radiopaque material, (d) only the suture 18 includes a radiopaque material, or (e) two or more of the components of the vascular closure device 10 may include a radiopaque material. Some examples of such vascular closure devices are disclosed herein. While the examples disclosed herein may include features/discussion pertaining to just one of the components of a given vascular closure device, it can be appreciated that these features (and the overall discussion) can be applied to any of the components of a vascular closure device, as appropriate, without departing from the spirit of the disclosure.

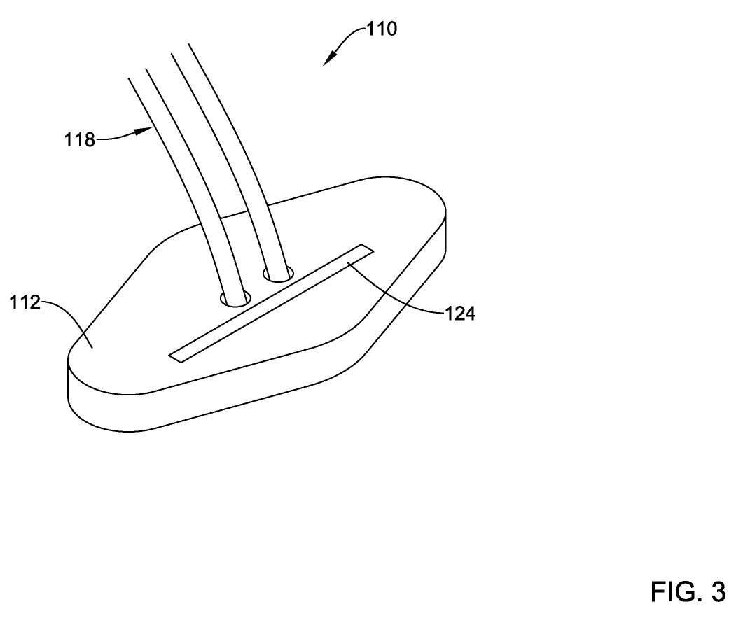

FIG. 3 illustrates a portion of another example vascular closure device 110. The vascular closure device 110 may be similar in form and function to other closure devices disclosed herein. For example, the vascular closure device 110 may include an intravascular sealing member 112 and a suture 118 coupled to the intravascular sealing member 112. The vascular closure device 110 may also include an extravascular sealing member (not shown) and/or an extravascular hemostatic member (not shown).

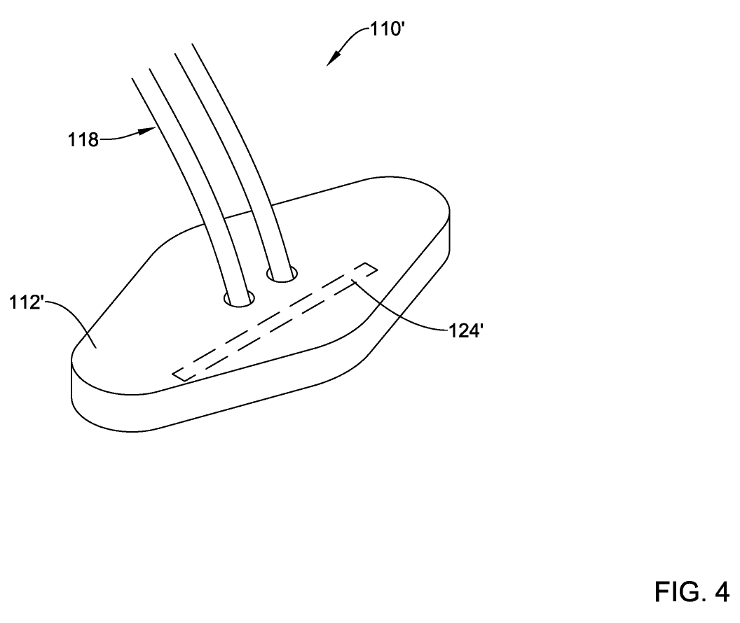

The vascular closure device 110 may include an imaging member 124 configured to enhance visualization thereof. In this example, the imaging member 124 may take the form of a radiopaque structure such as a wire that may be disposed along an exterior (e.g., top or blood vessel wall facing surface) surface of the intravascular sealing member 112 as shown in FIG. 3. FIG. 4 illustrates a variation of a vascular closure device 110’ where the intravascular sealing member 112’ includes an imaging member 124’ taking the form of a radiopaque structure such as a wire embedded within the intravascular sealing member 112’. Such imaging members 124, 124’ may help a clinician to visualize the intravascular sealing member 112, 112’, for example using conventional fluoroscopy. Other intravascular sealing members are contemplated where a radiopaque and/or echogenic material is loaded/doped therein. This may be in combination with the imaging members 124, 124’ or as an alternative to using the imaging members 124, 124’.

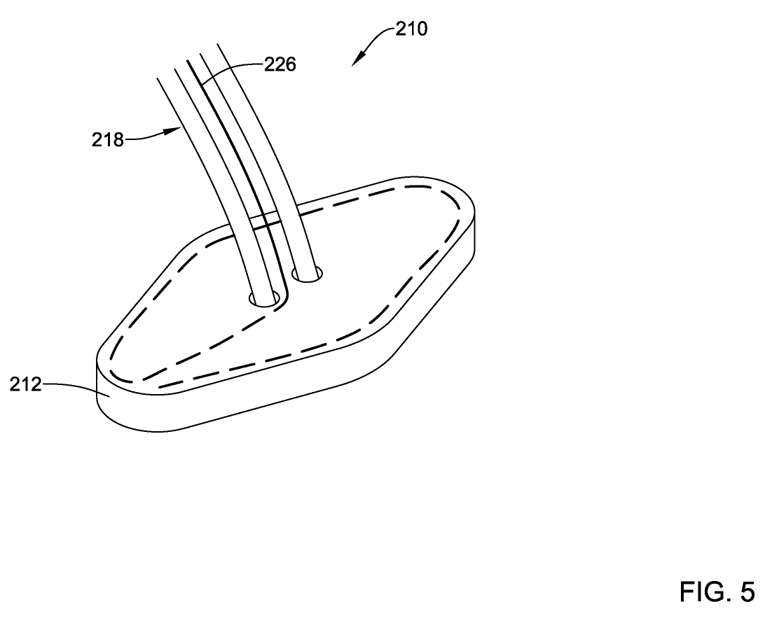

FIG. 5 illustrates a portion of another example vascular closure device 210. The vascular closure device 210 may be similar in form and function to other closure devices disclosed herein. For example, the vascular closure device 210 may include an intravascular sealing member 212 and a suture 218 coupled to the intravascular sealing member 212. The vascular closure device 210 may also include an extravascular sealing member (not shown) and/or an extravascular hemostatic member (not shown).

The vascular closure device 210 may include an imaging member 226 configured to enhance visualization thereof. In this example, the imaging member 226 may take the form of a radiopaque wire that is disposed within the intravascular sealing member 212. In some instances, the intravascular sealing member 212 may have a pre-formed channel or lumen formed therein for the imaging member 226 to extend within. The channel may extend about the perimeter or periphery of the intravascular sealing member 212 in any suitable manner. This may allow the imaging member 226 to be disposed along a greater/wider section of the intravascular sealing member 212. In some instances, the imaging member 226 may be fixed within the channel. In other instances, the imaging member 226 may be slidably or releasably disposed within the channel so that, for example, the imaging member 226 can be removed when the vascular closure device 210 has sufficiently managed/sealed/closed the opening into the vessel. In some instances, the imaging member 226 may extend externally of the intravascular sealing member 212 along the sutures 218. In other instances, the imaging member 226 may not extend externally of the intravascular sealing member 226 and be wholly contained within intravascular sealing member 226.

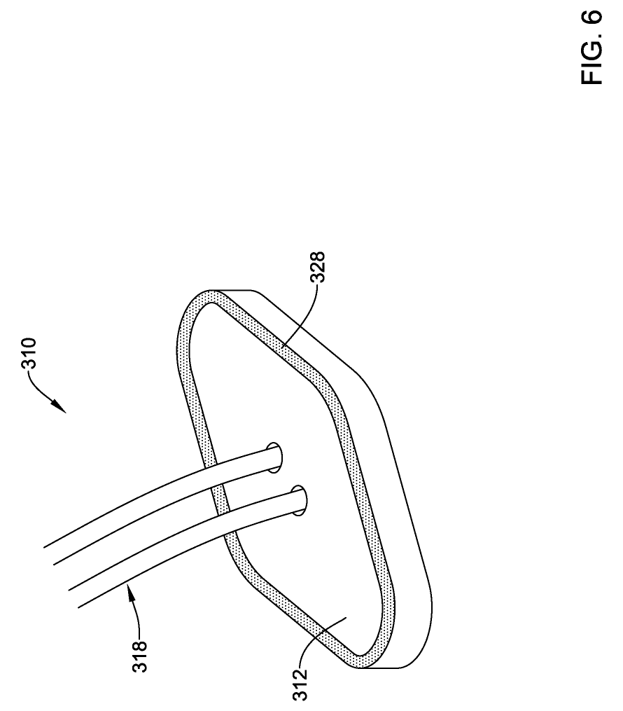

FIG. 6 illustrates a portion of another example vascular closure device 310. The vascular closure device 310 may be similar in form and function to other closure devices disclosed herein. For example, the vascular closure device 310 may include an intravascular sealing member 312 and a suture 318 coupled to the intravascular sealing member 312. The vascular closure device 310 may also include an extravascular sealing member (not shown) and/or an extravascular hemostatic member (not shown). The vascular closure device 310 may include an imaging member 328 configured to enhance visualization thereof. In this example, the imaging member 328 may include a chamber or lumen that can be filled with a radiopaque material. The chamber 328 may be “unfilled” prior to use. When ready for use, a clinician may inject or otherwise dispose a radiopaque material into the chamber 328. This may include injecting the radiopaque material through a port or opening (not shown) formed in the chamber 328, submersing the intravascular sealing member 312 into a container of contrast media so that the contrast media may enter/fill the chamber 328, and/or using another suitable methodology. In some instances, it may be desirable to allow the contrast media within the chamber 328 to at least partially dry or thicken, which may help to reduce loss of contrast media when coming into contact with water, saline, and/or blood.

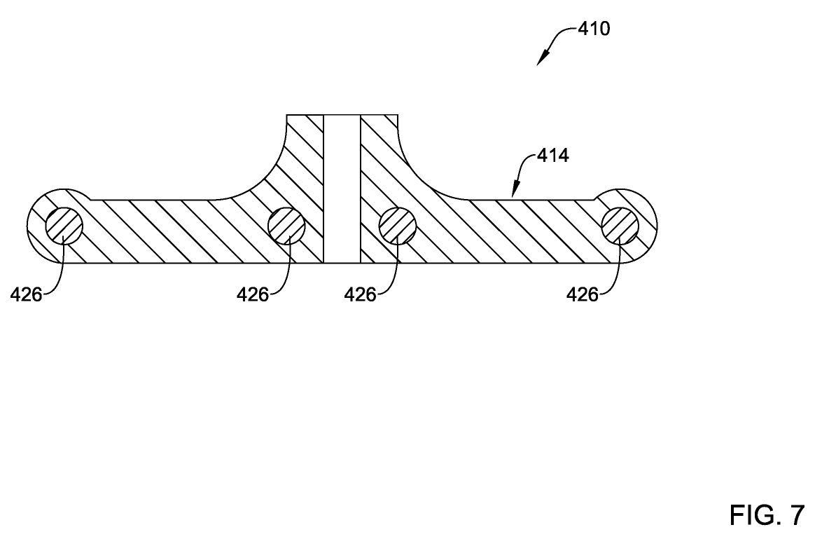

While incorporating imaging features into an intravascular sealing member may be an effective manner of enhancing visualization, other components may also include imaging features. For example, FIG. 7 illustrates a portion of another example vascular closure device 410, which may be similar in form and function to other closure devices disclosed herein. In FIG. 7, only the extravascular sealing member 414 is shown. The extravascular sealing member 414 may include an imaging member 426 configured to enhance visualization thereof. In this example, the imaging member 426 may take the form of a radiopaque wire that is disposed within the extravascular sealing member 414. The imaging member 426 may aid with alignment and/or confirmation that the extravascular sealing member 414 is position as desired with respect to the blood vessel (e.g., external to the blood vessel). The imaging member 426 may include suitable materials such as those disclosed herein. In at least some instances, the imaging member 426 may include iron, which may help to stiffen the extravascular sealing member 414 and/or help to enhance sealing.

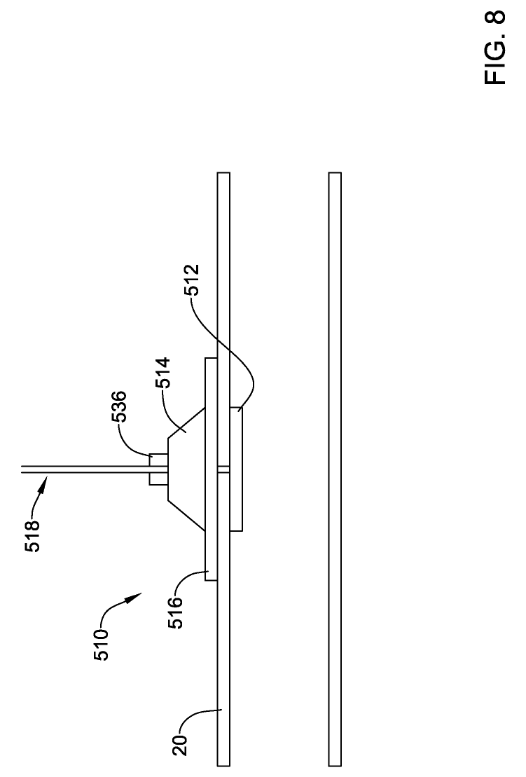

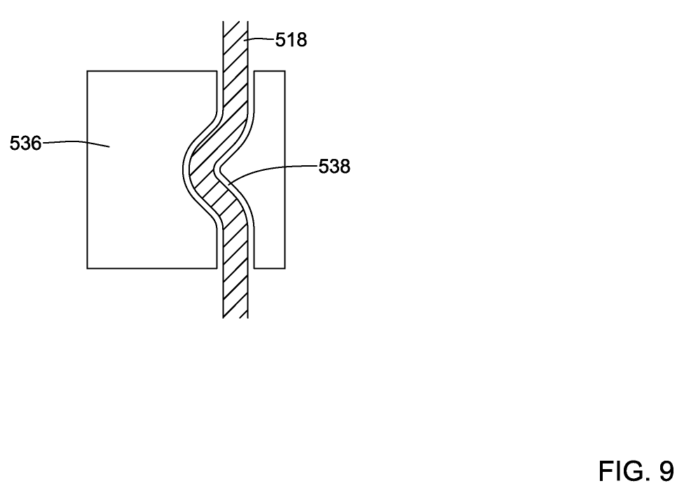

FIGS. 8-9 illustrate a portion of another example vascular closure device 510, which may be similar in form and function to other closure devices disclosed herein. The vascular closure device 510 may include an intravascular sealing member 512, an extravascular sealing member 514, and an extravascular hemostatic member 516. A suture 518 may be coupled to the intravascular sealing member 512 and/or the extravascular sealing member 514. In this example, a locking member 536 may be coupled to the suture 518 that allows the components (e.g., the intravascular sealing member 512 and the extravascular sealing member 514) to be secured relative to one another. As shown in FIG. 9, the locking member 536 may have a suture pathway 538 formed therein. The locking member 536 may be crimpable/pinchable to secure onto the suture 518. In at least some instances, the locking member may include a radiopaque material such as any of those disclosed herein.

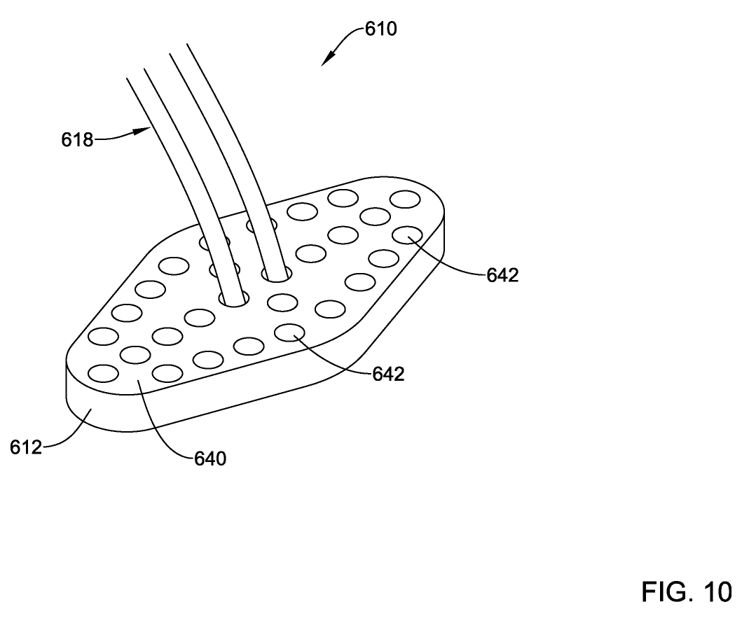

FIG. 10 illustrates a portion of another example vascular closure device 610. The vascular closure device 610 may be similar in form and function to other closure devices disclosed herein. For example, the vascular closure device 610 may include an intravascular sealing member 612 and a suture 618 coupled to the intravascular sealing member 612. The vascular closure device 610 may also include an extravascular sealing member (not shown) and/or an extravascular hemostatic member (not shown). The vascular closure device 610 may include an echogenic surface 640 having a plurality of imaging members or dimples 642 (e.g., inwardly-extending dimples, outwardly-extending dimples/projections/domes) formed therein that are configured to enhance visualization thereof. For example, the dimples 642 may help to scatter acoustic energy, which may allow for improved visualization using ultrasound. A number of patterns and/or configurations are contemplated. In some instances, the dimples 642 may have a size in the range of about 0.01-1.0 millimeters. The number of dimples may vary. In some instances, about 10-1,000 dimples 642 may be included. The dimples 642 can have any suitable shape such as spherical, triangular, rectangular, etc. The dimples 642 may be oriented inward and/or outward. Other echogenic structures may be utilized such as microspheres (e.g., bioresorbable spheres, glass spheres, etc.), textured surfaces, bubbles, pocked surfaces, and/or the like. In at least some instances, the echogenic surface 640 may face the wall of the blood vessel during use.

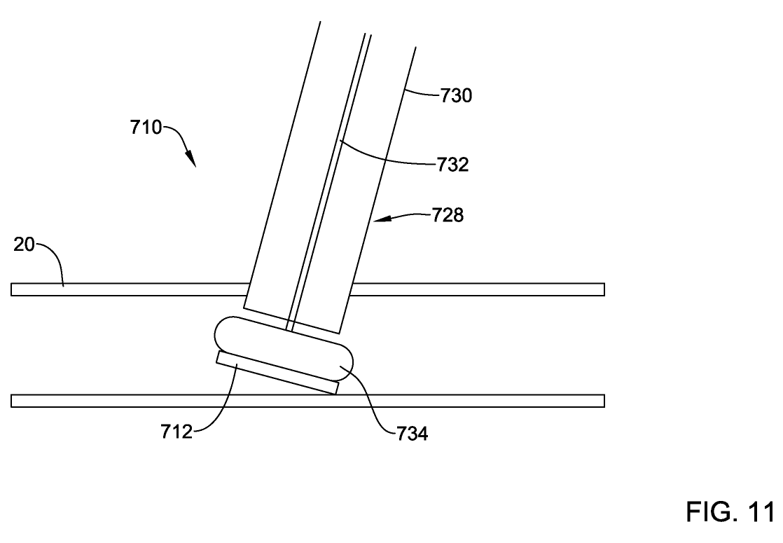

FIG. 11 illustrates a portion of another example vascular closure device 710, which may be similar in form and function to other closure devices disclosed herein. The vascular closure device 710 (as well as other vascular closure devices disclosed herein) may include and/or otherwise be used in conjunction with a delivery device 728. The delivery device 728 may include a delivery sheath 730 and a delivery arm or shaft 732.

The vascular closure device 710 may include an imaging member 734 configured to enhance visualization thereof. In this example, the imaging member 734 may include a balloon that may, for example, be coupled to the shaft 732 and disposed adjacent to an intravascular sealing member 712. As such, the delivery arm 732 may be used to inflate the balloon 734. The balloon 734 may be fillable with a radiopaque inflation media, which may allow for improved visualization during a vascular closure procedure.

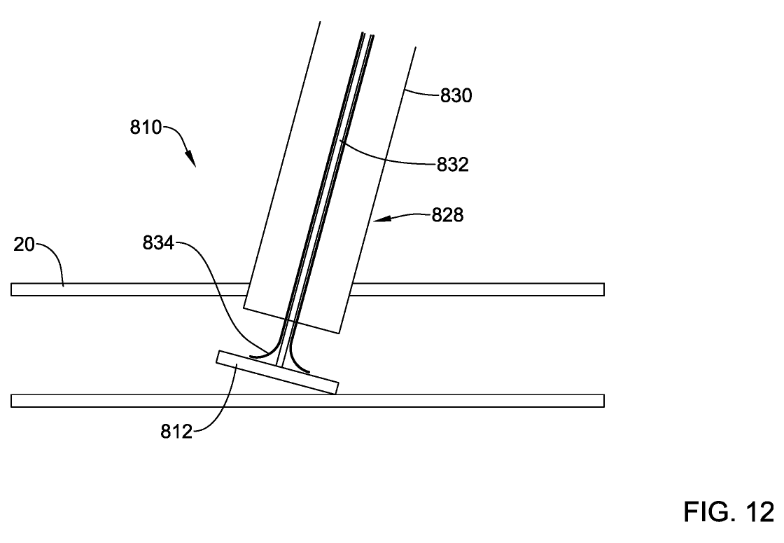

FIG. 12 illustrates a portion of another example vascular closure device 810, which may be similar in form and function to other closure devices disclosed herein. The vascular closure device 810 (as well as other vascular closure devices disclosed herein) may include and/or otherwise be used in conjunction with a delivery device 828. The delivery device 828 may include a delivery sheath 830 and a delivery arm or shaft 832.

The vascular closure device 810 may include an imaging member 834 configured to enhance visualization thereof. In this example, the imaging member 834 may include a radiopaque wire that extends along the delivery arm 832 and engages the intravascular sealing member 812.

The materials that can be used for the various components of the vascular closure device 10 (and/or other vascular closure devices disclosed herein) may include those commonly associated with medical devices. For simplicity purposes, the following discussion makes reference to the vascular closure device 10 and/or the components thereof. However, this is not intended to limit the devices and methods described herein, as the discussion may be applied to other devices/components disclosed herein.

The vascular closure device 10 and/or other components of the vascular closure device 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as VESTAMID®, GRILAMID® available from EMS American Grilon, and/or the like), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the vascular closure device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the vascular closure device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the vascular closure device 10 to achieve the same result.

In some embodiments, a degree of Magnetic Resonance Imaging (MRI) compatibility is imparted into the vascular closure device 10. For example, the vascular closure device 10, or portions thereof, may be made of a material that does not substantially distort the image and create substantial artifacts (e.g., gaps in the image). Certain ferromagnetic materials, for example, may not be suitable because they may create artifacts in an MRI image. The vascular closure device 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention’s scope is, of course, defined in the language in which the appended claims are expressed.

What is claimed is:

1. A vascular closure device, comprising:
an intravascular sealing member configured to be disposed within a blood vessel;
an extravascular hemostatic member configured to be disposed adjacent to an exterior of the blood vessel;
an extravascular sealing member configured to be disposed along an outer surface of the extravascular hemostatic member;
a suture coupled to the intravascular sealing member and the extravascular sealing member; and
wherein at least one of the intravascular sealing member, the extravascular hemostatic member, the extravascular sealing member, and the suture includes a radiopaque material.

2. The vascular closure device of claim 1, wherein the intravascular sealing member includes the radiopaque material.

3. The vascular closure device of claim 2, wherein the radiopaque material is disposed along an outer surface of the intravascular sealing member.

4. The vascular closure device of claim 2, wherein the radiopaque material is embedded within the intravascular sealing member.

5. The vascular closure device of claim 1, wherein the suture includes the radiopaque material.

6. The vascular closure device of claim 1, further comprising a delivery sheath and a delivery arm disposed within the delivery sheath for delivering the vascular closure device to a vessel opening.

7. The vascular closure device of claim 6, wherein the delivery arm includes a radiopaque material.

8. The vascular closure device of claim 6, further comprising a balloon coupled to the delivery arm.

9. The vascular closure device of claim 8, wherein the balloon includes a radiopaque material.

10. The vascular closure device of claim 1, further comprising a locking member coupled to the suture.

11. The vascular closure device of claim 10, wherein the locking member includes a radiopaque material.

12. A vascular closure device, comprising:
a suture;
an intravascular sealing member coupled to the suture, the intravascular sealing member configured to be disposed within a blood vessel;
an extravascular sealing member coupled to the suture, the extravascular sealing member configured to be disposed adjacent to an outer surface of the blood vessel;
an extravascular hemostatic member coupled to the suture and disposed between the intravascular sealing member and the extravascular sealing member; and
wherein the suture, the intravascular sealing member, or both include an imaging member configured to enhance visualization thereof.

13. The vascular closure device of claim 12, wherein the imaging member includes a radiopaque material disposed along the intravascular sealing member.

14. The vascular closure device of claim 12, wherein the imaging member includes a radiopaque material embedded within the intravascular sealing member.

15. The vascular closure device of claim 12, wherein the imaging member includes a radiopaque material incorporated into the suture.

16. The vascular closure device of claim 12, wherein the imaging member includes an echogenic material disposed along or embedded within the intravascular sealing member.

17. The vascular closure device of claim 12, wherein the imaging member includes an echogenic surface formed along the intravascular sealing member.

18. The vascular closure device of claim 12, wherein the imaging member includes a radiopaque balloon disposed adjacent to the intravascular sealing member.

19. The vascular closure device of claim 12, further comprising a radiopaque locking member coupled to the suture.

20. A system for closing a vascular opening, the system comprising:
a delivery sheath;
a delivery arm slideably disposed within the delivery sheath; and
a closure device coupled to the delivery arm, the closure device comprising:
a suture,
an intravascular sealing member coupled to the suture, the intravascular sealing member configured to be disposed within a blood vessel,
an extravascular sealing member coupled to the suture, the extravascular sealing member configured to be disposed adjacent to an outer surface of the blood vessel,
an extravascular hemostatic member coupled to the suture and disposed between the intravascular sealing member and the extravascular sealing member, and
wherein the suture, the intravascular sealing member, or both include an imaging member configured to enhance visualization thereof.
